(19) United States
(12) Patent Application Publication
(10) Pub. No.: US 2025/0057953 A1
(43) Pub. Date: Feb. 20, 2025

(54) ELECTROLYTE COMPOSITION FOR INTRODUCTION INTO A HUMAN DIGESTIVE TRACT, BALANCED IN MACROELEMENT COMPOUND AND PH WITH A MURAL CHYMUS OF INITIAL PARTS OF THE SMALL INTESTINE

(71) Applicants: Aleksei Vladimirovich Voronov, Moscow (RU); Mikhail Arkadievich Zorin, Moscow (RU)

(72) Inventors: Aleksei Vladimirovich Voronov, Moscow (RU); Mikhail Arkadievich Zorin, Moscow (RU); Vladimir Valerievich Kiselev, Moscow (RU)

(73) Assignees: Aleksei Vladimirovich Voronov, Moscow (RU); Mikhail Arkadievich Zorin, Moscow (RU)

(21) Appl. No.: 18/431,003

(22) Filed: Feb. 2, 2024

(30) Foreign Application Priority Data
Dec. 3, 2022 (RU) 2022131583

(51) Int. Cl. A61K 47/02; A61K 9/08

(52) U.S. Cl. CPC A61K 47/02; A61K 9/08

(57) ABSTRACT

The chyme-adapted saline electrolyte solution for administering enteral infusions includes sodium chloride, potassium chloride, sodium acetate, sodium phosphate, calcium chloride, magnesium sulfate, water at the following ions ratio, wt. %: sodium 12.4-35.4—potassium 2.5-15.6—calcium 0.65-7.785—magnesium 0.45-4.671—chlorine 20.4-48.65—phosphate 3.1-34.4—sulphate 0.28-13.27—acetate to 23.27—water the rest.

The present technical solution generally relates to a field of medicine, namely to an intensive care medicine, surgery, combustiology, traumatology, therapy, gastroenterology, oncology, oncohematology, dermatology, toxicology, gerontology, palliative, sports medicine, as well as in people whose occupation or lifestyle is associated with increased loss of water and electrolytes and can be used as a saline electrolyte solution for enteral infusion in the above-mentioned departments.

Many different variations of saline solutions are known in medicine, administered both parenterally and enterally. Enteral administration involves the oesophagus, stomach, small intestine and large intestine (i.e. the gastrointestinal tract). Methods of enteral administration also include oral, sublingual and rectal administration [2]. The use of stomas, e.g. gastrostomies, etc. for enteral administration can be distinguished separately.

Parenteral solutions are fluids used in intravenous therapy to restore or maintain normal fluid volume and electrolyte balance when oral administration is not possible. Intravenous infusion therapy is an effective and efficient way of delivering fluids directly into the vascular bed, to replenish electrolyte losses and administer drugs and blood products. There are different types of IV fluids and different ways of classifying them.

Developers of parenteral solutions used in medicine traditionally proceeded from the assumption that their ionic composition should be as close as possible to the macroelement composition of blood plasma and osmolarity. This is certainly justified when creating parenteral solutions, but the creation and application in practice perfectly balanced in macroelement composition with blood plasma parenteral crystalloid solution was difficult due to technical reasons. All parenteral electrolyte solutions are the result of the desire to maximise the macronutrient composition and osmolarity of the solution to the composition of blood plasma, but there are objective reasons why this is impossible.

Electrolyte solutions (“crystalloid solutions”) are used to compensate (in case of dehydration) or to cover fluid requirements as part of parenteral nutrition and to compensate for electrolyte disorders. Because of their low oncotic pressure, they remain only in the blood vessels for a short time and are distributed in the extracellular space, so they are only suitable to a very limited extent to compensate for large blood losses in hypovolaemic shock.

Because of the rapid redistribution, there is a risk of cerebral and pulmonary oedema if large volumes of solutions are administered [10]. Parenteral administration of electrolyte infusions also carries the following risks [5, 6, 7, 8, 9]:
1) the occurrence of infiltration;
2) the occurrence of a haematoma;
3) air embolism;
4) phlebitis and thrombophlebitis;
5) extravascular injection;
6) intra-arterial injection as a complication of intravenous injection;
7) hypersensitivity.

In addition, intravenous infusions in intensive care patients often cause water-electrolyte disturbances that are difficult to correct. All balanced electrolyte solutions currently in use represent a compromise between the actual electrolyte composition of human blood plasma and the requirements for electrolytes actually present in the infusion solution, which existing infusion solutions do not or only partially satisfy. The discrepancy between the ideal and actual composition is reinforced by the fact that any endeavour to respect the concentration of certain elements must necessarily lead to the neglect of other elements.

A classic example of the mismatch between the content of macronutrient ions in blood plasma and their content in the solution for intravenous infusion is the most commonly used in medical practice worldwide physiological solution of sodium chloride at a concentration of 0.9%. The adjective “physiological” in relation to this solution can be considered incorrect, because the chloride concentration in 0.9% NaCl is supraphysiological (154 vs. 100 mmol/l). A side effect of saline solution is hyperchloremic metabolic acidosis. The change in serum chloride and the volume of 0.9% NaCl administered in the study correlated with the degree of acidosis [12, 13, 14]. The use of saline solution after open abdominal surgery was associated with higher mortality and more infections, blood transfusions, renal replacement therapy (RRT), electrolyte disturbances and acidosis [15]. Ringer's solution, which is frequently used for parenteral administration, also has a similar chloride concentration to physiological saline.

The solution described in German patent DE102009012671A1 (BRAUN MELSUNGEN AG) published on 16 Sep. 2010 is known from the prior art. The indicated plasma-adapted “complete” electrolyte kit for parenteral administration, which, according to the authors, has significant advantages over traditional solutions, in particular, fewer complications.

The disadvantages of this solution are that this solution is proposed to be supplied in the form of dry components and diluted before application. This method of application indicates a short shelf life of the ready solution, in addition, this form of release is inconvenient for use. In addition, despite the long period of time that has passed since the patent was granted, the “complete” infusion solution has not appeared on the market, which May 25 indicate difficulties encountered in its production or clinical trials.

The patent of the Russian Federation RU2533257 C1 (State Budgetary Educational Institution of Higher Professional Education “St. Petersburg State Pediatric Medical University” of the Ministry of Health of the Russian Federation) published on 20 Nov. 2014 is also known from the prior art. The claimed solution for parenteral administration “Physiolite”, according to the authors, is balanced in the content of electrolytes and glucose, does not disturb the acid-base equilibrium and is optimal for intravenous replenishment of deficiency and provision of physiological need for water and basic electrolytes. The electrolyte solution comprises, in addition to macronutrient ions, glucose and fumarate.

The disadvantages of the above solution are that this solution cannot be considered complete in terms of macronutrients composition, as it lacks phosphorus and sulphur, macronutrients present in blood plasma, besides, parenteral route of solution administration, although traditionally used in medicine, is not physiological.

Meanwhile, the development of electrolyte solutions for enteral administration has not yet taken into account the possibility of creating a solution that is close in composition to the fasting chyme of the early small intestine. The World Health Organisation suggests the oral administration of glucose-salt solutions for the treatment of infectious diarrhoea. These oral rehydration solutions were developed without consideration of the macronutrient composition of the chyme, lack the following macronutrients: magnesium, calcium, phosphorus and sulphur, and are indicated solely for the treatment of diarrhoea. There are many oral rehydration solutions including brand names such as Ceralyte, Enfamil, Enfalyte, Pedialyte, Rehydron, etc. [1, 3, 4]. When rehydration solutions are used correctly, in diarrhoea, complications from their administration are not frequent and are limited to oedema and vomiting. In many cases, in infectious diarrhoea, the use of oral rehydration solutions can do without antibiotics.

The presence of sodium citrate in oral rehydration solutions designed to correct acid-base balance is not optimal due to the fact that the properties of sodium citrate include a decrease in blood coagulation, increase in the concentration of Na+ ions in plasma, which may adversely affect the condition of intensive care patients. The presence of glucose (or other sugars) in oral rehydration solutions in clinical cases not associated with infectious diarrhoea is undesirable, because in patients in critical condition, often catastrophic changes in the qualitative and quantitative composition of intestinal microflora, opportunistic microflora begins to prevail over normoflora, there is a process of colonization of the small intestine with microflora characteristic of the large intestine. An increase in the proportion of pathogenic microorganisms in the intestinal microbiota can lead to bacterial overgrowth syndrome (SIBOS) and translocation of microbial substrates into the lymphatic and bloodstream. Bacterial translocation leads to escalation of systemic inflammation and is a trigger for the development of sepsis and multi-organ dysfunction. Administration of a solution comprising glucose, which in this situation may be a nutrient substrate for bacteria, may contribute to further changes in the composition of the intestinal microbiota in the direction of an increase in the proportion of pathogenic microflora and aggravation of the general state of the macroorganism.

The general disadvantages of the solutions known from the state of the art are the impossibility of using electrolyte solutions alone to provide the physiological requirement of the human body in water and basic electrolytes. There is a practice of using parenteral saline solutions for enteral administration. Solutions for parenteral administration are not so effective for washing of enterocytes, stimulation of intestinal motility and absorption of nutrients in intestinal paresis and intestinal insufficiency syndrome in comparison with our proposed chyme-adapted enteral solution, imitating chyme by composition of macronutrients, pH and osmolarity. Also, the use of oral rehydration solutions is significantly limited in practice when treating severe patients, and is associated with:
1) the inconvenience of their preparation (as a rule, the solution is prepared immediately before application),
2) the tradition of intravenous administration of solutions in emergency situations,
3) the lack of practice in administering intestinal probes to the majority of intensive care patients who actually need it,
4) gastrointestinal motility disorders,
5) absorption disorders,
6) danger of regurgitation of gastric contents with the risk of aspiration of the tracheo-bronchial tree and subsequent development of pneumonia,
7) underestimation by the medical community of the importance of preventing small intestinal ischaemia, as well as motor, absorptive disorders and translocation of intestinal microflora resulting from ischaemia of the small intestinal wall.

Restricting the use of oral rehydration solutions in patients who do not require resuscitation care increases the cost to the health care system of intravenous infusions and the management of complications associated with them.

This technical solution is aimed at eliminating the disadvantages inherent in the existing solutions known from the prior art.

The technical problem in this technical solution is to create a new and effective saline electrolyte solution for enteral infusions.

The main technical result, manifested by solving the above problem, is to improve the efficiency of regulation of water-electrolyte balance in patients during enteral infusions.

An additional technical result, manifested in solving the above problem, is a reduction in the level of endogenous intoxication during enteral infusions with saline electrolyte solution.

The claimed technical results are achieved by realizing a saline electrolyte solution for carrying out enteral infusions, comprising in its basic composition:
sodium chloride, potassium chloride, sodium acetate, sodium phosphate, calcium chloride, magnesium sulfate, water at the following ions ratio, wt. %:
   sodium 12.4 to 35.4;
   potassium 2.5-15.6;
   calcium 0.65-7.785;
   magnesium 0.45-4.671;
   chlorine 20.4-48.65;
   phosphate 3.1-34.4;
   acetate to 23.27;
   sulphate 0.28 to 13.27.

In one particular embodiment, the saline electrolyte solution additionally comprises citric acid or salts thereof, wherein the citrate is comprised in the following ions ratio: 0.08-19.45 wt. %.

In another particular embodiment, the saline electrolyte solution further comprises at least one acid from the group of tricarboxylic acids, and/or salt(s) of tricarboxylic acids, which is preferred.

In another particular embodiment, the saline electrolyte solution further comprises at least one of the group consisting of: vitamins, vitamin derivatives, vitamin-like substances, micronutrients, probiotics, prebiotics, metabiotics, peptides, lipids.

In another particular embodiment, the saline electrolyte solution further comprises amino acid(s), salts of amino acids, and derivatives of amino acids.

In another particular embodiment, the saline electrolyte solution additionally comprises gluconate at the following ion ratio, wt %: gluconate 0,05-18,2.

In another particular embodiment, the saline electrolyte solution is characterised in that the vitamins are water-soluble vitamins, fat-soluble vitamins or a combination thereof.

In another particular embodiment, the saline electrolyte solution further comprises medium-chain triglycerides.

In another particular embodiment, the saline electrolyte solution further comprises glucose, fructose, sucrose.

In another particular embodiment, the saline electrolyte solution further comprises acetic acid or salts thereof.

In another particular embodiment, the saline electrolyte solution further comprises butanoic acid, monobutyrin, dibutyrin, tributyrin, or salts thereof.

In another particular embodiment, the saline electrolyte solution further comprises propionic acid or salts thereof.

In another particular embodiment, the saline electrolyte solution further comprises isobutyric acid or salts thereof.

In another particular embodiment, the saline electrolyte solution further comprises valerian acid or salts thereof.

In another particular embodiment, the saline electrolyte solution further comprises isovaleric acid or salts thereof.

In another particular embodiment, the saline electrolyte solution further comprises lactic acid or salts thereof.

In another particular embodiment, the saline electrolyte solution further comprises maleic acid or salts thereof.

In another particular embodiment, the saline electrolyte solution further comprises bacteria, including bifido-, lacto-, coli-bacteria, bacteria products, or combinations of compositions of bacteria and bacteria products.

In another particular embodiment, the saline electrolyte solution further comprises lactulose.

In another particular embodiment, the saline electrolyte solution further comprises Hermetia illucens fat or Hermetia illucens extract.

In another particular embodiment, the saline electrolyte solution further comprises an ozone solution.

In another particular embodiment, the saline electrolyte solution further comprises succinic acid or salts thereof.

In another particular embodiment, the saline electrolyte solution further comprises pharmaceutical compositions.

In another particular embodiment, the saline electrolyte solution further comprises molecular or atomic hydrogen.

The claimed technical results are also achieved by realising a method of administering a saline electrolyte solution, comprising administering a saline electrolyte solution.

In one particular embodiment, the administration is via one method selected from the group consisting of enteral infusion, GI lavage, siphoning, drinking, enema, via stoma or drainage, and graft washout.

The claimed technical solution allows achieving optimal results in enteral infusion therapy and treatment of diseases associated with gastrointestinal tract dysfunction (GI paresis, intestinal failure syndrome, ulcerative colitis, Crohn's disease, antibiotic-associated colitis, etc.), preparation of patients for surgeries, primarily surgeries on gastrointestinal tract organs or surgeries using artificial circulation, due to the created saline electrolyte water solution (abbreviated as SES), which is an analogue of the premenopausal chyme of the initial parts of the jejunum, and its use can be carried out both independently and as a carrier for various nutrients, vitamins, trace elements, probiotics, prebiotics, metabiotics, amino acids, peptides, lipids, etc., or combinations thereof, or pharmaceutical compositions. This is due to the fact that enteral administration of liquids and substrates of the micronutrient component of chyme is the only physiological, it is from the digestive system that the process of regulation of water-electrolyte metabolism, acid-base balance, energy metabolism, nutrient intake in the human body begins.

Since SES is chyme-adapted, i.e. by ionic composition and pH is as close as possible to the fasting chyme of the jejunum, its administration has a favourable effect on the state and function of the intestinal wall. Administration of SES normalises gastrointestinal motility, improves intestinal absorption, and prevents increased permeability of the intestinal barrier and microflora translocation. As the results of sequencing the microbiota of the initial parts of the small intestine in patients with pancreonecrosis showed, a more severe course of the disease was associated with reduced representation in the microbiome of Nesseria mucosa and Parvimonas micra species inhabiting the mucosal layer, as well as Megasphaera micronuciformis. The proportion of Streptococcus genera (S. rubneri/parasanguinis/australis species) and Actinomyces, as well as a number of genera from the family Enterobacteriaceae in such patients, on the contrary, was higher. The study obtained data that in patients with pancreonecrosis who received SES from day 1 of the disease, there is no significant decrease in the species of Nesseria mucosa, Parvimonas micra and Megasphaera micronuciformis, as well as an increase in the proportion of Streptococcus genera (S. rubneri/parasanguinis/australis species), Actinomyces, genera of the family Enterobacteriaceae, in contrast to patients who did not receive SES in the first 24-48 hours from the moment of the disease [17].

It is known that changes in the composition of intraluminal fluid (chyme) comprised in the small intestine is one of the elements of triggering the process of intestinal failure syndrome development [16]. Table 1 shows the most optimal parameters of SES compared to the parameters of the jejunal premenopausal chyme and blood plasma.

TABLE 1

| | Content in SES, | Chyme | Content in |
|---|---|---|---|
| | most optimal, | content, | blood plasma, |
| Name | mmol/L | mmol/L | mmol/l |
| Sodium | 84.0-136.0 | 95.6 | 130-156 |
| Potassium | 15.8-31.3 | 20.5 | 3.4-5.3 |
| Calcium | 4.8-12.2 | 7.5 | 2.3-2.75 |
| Magnesium | 2.5-8.9 | 6.6 | 0.7-1.2 |
| Phosphorus | 7.2-19.0 | 15.6 | 1 |
| Chlorine | 86.1-132.0 | 98.6 | 97-108 |
| RN | 4.45-6.4 | 5.5-5.8 | 7.35-7.45 |

At the same time, it should be taken into account that, unlike electrolyte solutions administered parenterally, SES allows to achieve the necessary therapeutic effects with a wider variation of osmolarity and PH parameters of the solution administered in the GI tract. This is due to the fact that in the human body the only natural way to obtain water and electrolytes is their intake through the GI tract. Accordingly, the natural process of homeostasis of both small intestinal chyme and blood plasma is activated during the introduction of SES. It should be noted that gastric mucosa plays an active role in the process of chyme homeostasis, therefore, for administration directly into the small intestine via a probe in intensive care patients, it is necessary to take into account more strictly the ratio of parameters of administered SES and small intestinal wall chyme. If the patient has gastrostasis or there is a threat of compression of the initial parts of the small intestine by an inflammatory infiltrate, for example, in pancreonecrosis, it is necessary to use a nasointestinal tube to administer SES by the ligament of Treitz.

As practice shows, to normalise GI function it is sufficient to administer SES in the form of a basic composition or SES with the addition of easily digestible nutrients (monomers) in the small intestine. Normalisation of GI motility when administering SES is associated with the fact that one of the mechanisms of regulating the level of ions and nutrients entering the blood plasma through the intestinal wall is a change in intestinal motility.

Next, let us consider the main substances that can be used to prepare 1 litre of SES. In some cases, the solution composition can be made without one or more of the substances listed in Table 2. The substances used are listed in Table 2.

TABLE 2

| Substances | Quantity, g. |
|---|---|
| Sodium chloride | up to 5.1 |
| Sodium acetate, sodium acetate | to 5.78 |
| trihydrate, sodium diacetate | |
| Potassium acetate | to 4.16 |
| Potassium chloride | to 2.83 |
| Potassium phosphoric acid single- | to 5.91 |
| substituted, potassium phosphoric | |
| acid double-substituted three-water | |
| Sodium phosphoric acid, sodium | to 5.21 |
| phosphoric acid duodeca or | |
| anhydrous, two or three times | |
| substituted | |
| Calcium chloride, calcium chloride | to 3.9 |
| hexahydrate | |
| Magnesium sulphate, magnesium | up to 3.3 |
| sulphate seven-water | |
| Magnesium chloride | up to 1.8 |
| EDTA | up to 2.6 |
| Citric acid anhydrous or | up to 1.5 |
| monohydrate | |
| Sodium citrate | up to 2.4 |
| Potassium citrate | up to 3.25 |
| Water | up to 1 litre |

The claimed SES can be introduced into a body, for example, by following methods: drinking, siping, introduction into the stomach by probe or endoscope channel, introduction into the small intestine by probe or endoscope channel, introduction through a stoma, rectally in an enema, washing of cavities and hollow organs, washing of the lumen of the intestinal graft at the time of taking the donor organ and during preparation of the graft immediately before transplantation. The most commonly used volume and rate of administration of SES: drinking on an empty stomach 0.5-2.5 litres in portions (from 1 to 20 intakes per day), sipping from 10 to 50 ml at a time, volume 0.5-2 litres per day, enteral infusion at a rate from 2 to 35 ml per minute, gastrointestinal lavage in the natural direction-single volume not less than 4 litres, rate not less than 2 litres per hour, rectal administration-single volume from 10 ml to 2 litres, rate arbitrary. Classification of applied SES by osmolarity: hypoosmolar, hyperosmolar, isoosmolar.

The claimed SES can be categorized by sterility: sterile and non-sterile. Since SES is administered in the GI tract, it may be prepared at the patient's bedside from concentrates and not be sterile. At the same time, a sterile set of concentrates or solutions, including the prepared solution, allows SES to be stored for a longer period of time, and sterile solutions are preferred for resuscitation patients.

Purposes of SES use: as a solution to restore the volume of circulating blood, regulation of water-electrolyte balance and acid-base state, as a carrier solution for drug components, to normalise GI motility, to improve the state of enterocytes (washing of enterocytes, normalisation of absorption), prevention of microflora translocation, preparation of the patient's intestine for enteral nutrition.

Preferred embodiments of the balanced SES, which are intended primarily for administration by probe directly into the jejunum at least 30 cm beyond the ligament of Treitz, according to the invention, comprise the macronutrient ions sodium, potassium, calcium, magnesium, sulphur, phosphorus, carbon, chlorine and independently correspond to at least one of the following concentrations:
a) 82 to 156 mmol/l sodium, and
b) 10.1 to 37.96 mmol/l potassium, and
c) 2.64 to 17.53 mmol/L calcium, and
d) 3.06 to 16.85 mmol/l magnesium, and
(e) 85.1 to 144.67 mmol/L chlorine, and
(e) 4.19 to 43.41 mmol/L phosphate, and
(e) 2.71 to 42.49 mmol/L acetate.

In one embodiment, the SES may comprise citrate up to 10.75 mmol/L.

Furthermore, the SES preferably has a pH in the range from 4.45 to 6.8, more preferably from 5.0 to 6.4. The theoretical osmolality of the SES (comprising only electrolytes, without addition of additional substances) according to the invention is from 205 to 430 mosmol/kg, preferably a theoretical osmolality from 230 to 350 mosmol/kg.

The described advantages and features of the claimed technical solution are disclosed in more detail with reference to the drawings and the description set forth below. The above description is provided as an example and does not limit the scope of application and use of the claimed technical solution.

Next, let us consider the preferred forms of release of the claimed SES.

Forms of SES issuance may be, for example:
a) prepared solution,
b) a solution consisting of two or more components (comprising in vials, packets, etc.) mixed before use without or with the addition of water or other aqueous solutions not included in the SES kit,
c) a concentrate comprising two or more components (vial, packet) diluted in water, preferably deionized, distilled or soft water shortly before use,
d) a concentrate comprising dry, anhydrous components or euctic liquid having two or three or more components (vial, packet) diluted in water, preferably deionized, distilled or soft water, shortly before use.

The most preferred embodiments for use are embodiments b) and d) described above.

Variant a) has a relatively short shelf life, variant b) is convenient because the shelf life is longer than that of variant a) and the solution preparation process can be facilitated by packing the SES components into two, three or four-chamber solution bags [12].

Option d) is convenient because it has a long (up to 5 years) shelf life, minimal volume and weight, it is easy to store and transport, as the water used as a solvent can be found at the place of application.

Option c) is intermediate between options b) and d), has less usability than option b), has a higher mass and volume than option d).

Furthermore, said SES can be preserved, for example by means of the following methods:
radiation sterilization with ionizing radiation,
ozonization,
adding to the solution:
   hydrogen peroxide,
   sorbic acid and its salts (E200-E203),
   benzoic acid and its derivatives (E 210-219),
   preservatives comprising sulphur (E 221-228), nisin (E 234) and natamycin (E 235),
   acetic acid and acetates (E 260-266),
   nitrites and nitrates (E 249-252),
   Lactic acid and its derivatives—propionates (E270, E280-283), dimethyl carbonate, formic, malic and fumaric acids,
carbon dioxide treatment,
hforzr 2×tirzr jyn˜qjsjirfr rsjyjyjyffhjyfyj twJIYF (E385),
autoclaving,
pressurised steam treatment,
fluid steam treatment,
boiling,
pasteurisation.

It is worth noting that dry concentrate (or concentrate comprising salts in melt), may be produced in individual containers, jars, bottles, bags comprising the following concentrates:
1) sodium chloride, sodium acetate, potassium chloride, sodium phosphate, other components,
2) magnesium sulphate,
3) calcium chloride.

Any components may also be added to containers comprising magnesium sulphate and calcium chloride. In addition, the components may be added to one or more individual vials (bags) or mixed with one of the three concentrates above.

Liquid concentrates comprising solutions of salts in water are available in individual containers, jars, bottles, pouches or in packets comprising multiple chambers, combined before use and comprising:
1) sodium chloride, sodium acetate, potassium chloride, sodium phosphate, other components,
2) magnesium sulphate,
3) calcium chloride.

Any components may also be added to containers comprising magnesium sulphate, calcium chloride or solutions thereof. In addition, the components may be added to one or more individual vials (packets) or mixed with one of the three concentrates above.

Liquid solutions ready for use after mixing: their composition is similar to the above. The difference is that it is not necessary to add water, as its volume is optimized in the vials (bags) comprising it:
1) sodium chloride, sodium acetate, potassium chloride, sodium phosphate, other components,
2) magnesium sulphate,
3) calcium chloride.

Any components may also be added to containers comprising magnesium sulphate, calcium chloride or solutions thereof. In addition, the components may be added to one or more individual vials (bags) or mixed with one of the three concentrates mentioned above. In this case, some of the components may be in the form of concentrates, dry or solutions.

Also, in one particular embodiment, molecular or atomic hydrogen may be added to the saline electrolyte solution (made in advance in a manufacturing facility or made immediately prior to administration to the patient).

In yet another particular embodiment, vitamins, such as water-soluble vitamins, fat-soluble vitamins, or combinations thereof, may be added to the SES as described above.

In addition, the following components may also be added to the SES: medium-chain triglycerides, glucose, fructose, sucrose, butanoic acid or salts thereof, propionic acid or salts thereof, bifido, lacto, coli bacteria, products thereof or a combination thereof, lactulose, Hermetia illucens fat, ozone solution, succinic acid, derivatives thereof, incl. meglumine sodium succinate, etc. including meglumine sodium succinate, etc., not limited to.

According to the present invention, in the case of the manufacture of a medicament, salts included in relevant pharmacopoeias such as the European Pharmacopoeia, the United States Pharmacopoeia and the like are used. In a preferred embodiment of the invention, salts are used which are selected from the group consisting of: sodium chloride, sodium hydrophosphate, sodium salt of D-gluconic acid, potassium chloride, potassium acetate, sodium acetate, calcium D-gluconate and magnesium chloride, magnesium sulfate.

For preparation of SES solution for nutritional support it is also possible to use salts that are food additives, for example: sodium chloride, potassium chloride, sodium acetate, calcium chloride, magnesium sulphate, etc.

In addition, the claimed invention further relates to a method of producing an SES according to the invention, comprising producing a solution in a mould:
a) prepared solution,
b) a solution consisting of two or three or more components (vials, packets) mixed before use without adding water or other aqueous solutions not included in the SES kit;
c) a concentrate comprising two or more components (packed in vials, bags, etc.) diluted in water, preferably deionized, distilled or soft water before use;
d) a concentrate comprising dry, anhydrous components or euctic liquid, having two or more components (vial, bag) diluted in water, preferably deionized, distilled or soft water before use, as well as bringing the solution to a pH value in the required range;
e) capsules, tablets, including soluble (in water).

Due to its balanced and close macronutrient composition to chyme, good tolerability, the SES according to the present invention is very suitable as a solution for washing the GI tract naturally, for administration into the colon as an enema, for washing the intestine through stomas, for washing the patient's GI tract and transplanted intestinal sections both before and after transplantation.

The balanced composition of SES allows to prepare the intestines of patients before surgeries and colonoscopy by drinking or administering SES through a probe (gastric or intestinal) at a rate of at least 1.3 litres per hour in a single volume of 3 litres and more (up to pure lavage water).

Since SES by its macronutrient composition and pH is an analogue of jejunal chyme, it effectively triggers intestinal motility in paresis and intestinal failure syndrome, which allows to significantly accelerate the start of enteral nutrition. The use of SES to trigger intestinal motility in paresis or intestinal failure syndrome is of critical therapeutic importance for the prevention of infectious complications in intensive care patients. Comparative efficacy of SES application to trigger small intestinal motility in developing intestinal failure syndrome after gastric, small and large intestine surgeries is shown in Table 3.

TABLE 3

| Name | Main group | | Comparison group | |
|---|---|---|---|---|
| Appearance of | 36.0 (±15.6) | minutes | 375.0 (±207.2) | minutes. |
| instrumentally | | | | |
| confirmed signs of | | | | |
| bowel function | | | | |
| Independent stool | 1.05 (±0.2) | hours | 27.2 (±16.5) | hours |

| | | | |
|---|---|---|---|
| Enteral nutrition | 11 (±5.7) | hours | 29.5 (±16.5) |

The study included 20 patients (16 men and 4 women) with intestinal failure syndrome. The patients were divided into two groups: main and comparison, comparable in age, sex and severity of condition. In the main group, 4 to 4.5 litres of SES was administered by nasointestinal tube to stimulate the intestine; in the comparison group, traditionally used methods of intestinal motility stimulation were used.

The main group consisted of 8 men and 2 women, SES was administered to stimulate intestinal motility. The mean age was 45.9(±13.8) years. The mean APACHE II score was 8.5(±4.6) and SOFA score was 2.7(±1.3). The patients' diagnoses were pancreonecrosis, acute odontogenic brain abscess, ruptured abdominal aortic aneurysm, positional compression syndrome, cerebral infarction.

The comparison group consisted of 8 men and 2 women, and conventional therapy, without SES, was used to stimulate intestinal motility. The mean age was 46.6(±12.5) years. The mean APACHE II score was 8.9 (±4,4) points and SOFA score was 2.9(±1.8) points. The patients' diagnoses were pancreonecrosis, cerebral infarction, and craniocerebral trauma.

In the main group, the mean value of time interval from the beginning of SES administration to the appearance of instrumentally confirmed signs of bowel function was 36.0(±15.6) minutes, independent stool was obtained after 1.05(±0.2) hours. Enteral nutrition was started after 11(±5.7) hours. No complications related to the administration of SES were noted.

In the comparison group, the mean time interval from bowel motility stimulation to the appearance of instrumentally confirmed signs of bowel function was 375.0(±207.2) minutes, and independent stools were obtained after 27.2(±16.5) hours. Enteral feeding was started after 29.5(±16.5) hours.

Thus, the introduction of SES allowed more than 10 times faster recovery of intestinal motility and 2.6 times faster initiation of enteral nutrition in patients.

Based on the properties of the proposed SES, it can be used to treat hypotonic or isotonic dehydration, to treat extracellular fluid loss, hypovolemia or shock, and to rehydrate the interstitial space. The possibility of using SES as a carrier solution for nutrients and drugs compatible with the electrolytes it comprises must be answered separately.

One of the directions of SES application is changes in blood parameters after acute myocardial infarction. We present the data of laboratory measurements of blood parameters in 57 patients who took SES in the volume of 1.5 to 2 litres daily after acute myocardial infarction, from 1 to 7 days (Table 4). As the above data show, the use of SES in this category of patients is effective and safe.

The following is Table 4, showing the effect of daily administration of 1.5 to 2 litres of SES per day on laboratory parameters in patients after acute myocardial infarction, between days 1 and 7 (number of patients 57).

TABLE 4

| | 1 | 2 | 3 | 4 | 5 | 6 | 7 |
|---|---|---|---|---|---|---|---|
| Indicators | day | days | days | day | days | day | day |
| WBC | 2.98 | 14.09 | 19.65 | 13.97 | 12.04 | 9.38 | 9.0 |
| Procalcitonin, | 1.04 | 2.15 | 3.69 | 2.85 | 1.08 | 0.77 | 0.43 |
| ng/ml | | | | | | | |
| SRB, | 21 | 35 | 147.4 | 133 | 112.2 | 76 | 58 |
| mg/l | | | | | | | |
| Potassium | 3.3 | 2.4 | 3.7 | 3.5 | 3.3 | 3.5 | 3.7 |
| Sodium | 134 | 130 | 135 | 139 | 137 | 138 | 143 |
| Magnesium | 0.78 | 0.69 | 0.71 | 0.82 | 0.84 | 0.81 | 0.88 |
| Calcium | 1.01 | 1.04 | 1.05 | 1.08 | 1.1 | 1.13 | 1.14 |
| Ph | 7.39 | 7.38 | 7.34 | 7.35 | 7.39 | 7.40 | 7.43 |
| pcO2 | 25.8 | 24.5 | 28.9 | 32.2 | 35.6 | 38.4 | 44.2 |
| pO2 | 124 | 137 | 108 | 104 | 99 | 84 | 95 |

The effect of administering SES in combination with, for example, amino acids (e.g. glutamine or its salts) in enteral nutrition is due to normalization of intestinal motility and absorption. The same applies to the use of medications, for example, in anastomositis, e.g., of the jejunum or ileum. Anastamositis contributes to the development of intestinal paresis, which excludes the possibility of delivery of hormonal drugs (e.g. dexamethasone, which also has the property of inhibition of intestinal peristalsis) when taken orally to the site of inflammation. After triggering intestinal motility with SES, the second step is to administer SES with dexamethasone solution added to it in the concentration required to achieve the therapeutic effect.

Taking into account the fact that the mucosal layer is not a water-soluble substance and is the main component of the intestinal barrier environment, it is possible to use SES as a component to correct the intestinal microbiome, especially in conditions accompanied by SIBOS. A study describing the composition of the small intestinal microbiota in patients with acute pancreatitis was conducted on this topic. In this study, the bacterial composition of jejunal flush samples was investigated using 16S RNA gene sequencing. A more severe course was associated with a reduced representation in the microbiome of Nesseria mucosa and Parvimonas micra species inhabiting the mucosal layer, as well as Megasphaera micronuciformis. Proportion of Streptococcus genera (species S. rubneri/parasanguinis/australis) and Actinomyces and a number of genera from the family Enterobacteriaceae were, on the contrary, higher in such patients. Comparison of samples of patients with severe course against enteral infusion using saline enteral solution (SES), taken 24 hours apart, showed dynamics in favor of the microbiota of patients with a milder course of the disease. The results suggest an association between complications of acute pancreatitis and thinning of the jejunal mucosa. Enteral infusion of SES leads to a decrease in SIBOS and restoration of intestinal microbial passage towards an increase in microbial diversity [17].

Thus, embodiments of the present invention may be used, for example, in the following fields:
treatment of intestinal paresis, intestinal failure syndrome,
hypo- and hypermotility,
non-productive bowel motility in inpatients,
prevention of translocation and nosocomial infections, including pneumonia for therapy of pulmonary oedema, cerebral oedema,
preparation for operations,
preparation for and use after faecal transplantation,
restoration of the absorptive function of the intestine,
preparing for an early start of enteral nutrition,
water-electrolyte disorders, including but not limited to in infectious diseases, including but not limited to in intensive care patients, for the treatment of hypotonic or isotonic dehydration, for the treatment of loss of extracellular fluid, for the treatment of hypovolaemia or shock and for rehydration of the interstitial space,
replenishment of circulating blood volume (abbreviated as CBC),
colitis of infectious and autoimmune nature,
withdrawal from withdrawal syndrome of alcoholics and drug addicts,
washing of donor organs, including small and large intestine before transplantation,
detoxification and normalization of bowel function after chemotherapy,
phosphorus deficiency after chemotherapy, gastric and intestinal lavage through stomas and drains, if present,
enteral dialysis,
prevention and treatment of autoimmune diseases,
irritable bowel syndrome,
bacterial overgrowth syndrome (SIBOS),
constipation of medical genesis (against the background of antibiotics, psychotropic drugs),
as a means of delivering medicinal and nutritional components into the intestinal lumen and through the intestinal wall,
as a means of delivery to the colon (e.g. in colitis),
weight loss,
application in sports,
use after myocardial infarction to normalise water-electrolyte balance and blood pH.

### SOURCES OF INFORMATION USED

1. https://www.who.int/maternal_child_adolescent/documents/a85500/en/2
2. https://www.britannica.com/science/drug-chemical-agent/Functional-macromolecules #ref126883
3. https://medicalmed.us/regidron.htm
4. https://www.drugs.com/ppa/oral-rehydration-therapy.html
5. https://www.epainassist.com/opioid-treatment/medications/common-complications-associated-with-iv-therapy
6. (2008). I.V. Essentials: Complications of Peripheral I.V. Therapy. Nursing Made Incredibly Easy!, 6 (1).
7. Smeltzer, S. (2010). Brunner and Suddarth's Textbook of Medical Surgical Nursing, 12e. Philadelphia: Wolters Kluwer Health/Lippincott Williams & Wilkins.
8. Spencer, S. & Gilliam, P. (2015). Teaching patients about their short peripheral I.V. catheters. Nursing 2015, 45 (2).
9. Vacca, V. (2013). TIME CRITICAL: Vesicant extravasation. Nursing 2013, 43 (9
10. https://patents.google.com/patent/CH706750A2/de
11. https://www.renolit.com/en/industries/medical-pharmaceutical/nutrition
12. https://ncbi.nlm.nih.gov/pmc/articles/PMC5506238/13.
Reid F, Lobo D N, Williams R N, Rowlands B J, Allison S P. (Ab) normal saline and physiological Hartmann's solution: a randomised double-blind crossover study. Clin Sci (Lond) 2003; 104 (1): 17-24.
14. Waters J H, Miller L R, Clack S, Kim J V. Cause of metabolic acidosis in prolonged surgery. Crit Care Med. 1999; 27(10): 2142-2146. doi: 10.1097/00003246-199910000-00011.
15. Shaw A D, Bagshaw S M, Goldstein S L, et al. Major complications, mortality, and resource utilisation after open abdominal surgery: 0.9% saline versus Plasma-Lyte. Ann Surg. 2012; 255 (5): 821-829. doi: 10.1097/SLA.0b013e31825074f5.
16. Galperin Y. M., Lazarev P. I. Digestion and homeostasis. Moscow. Nauka. 1986. 304 c.
17. https://www.medrxiv.org/content/10.1101/2021.08.24.21262159v1

1. A saline electrolyte solution for administering enteral infusion, comprising:
wherein the saline electrolyte solution comprises sodium chloride, potassium chloride, sodium acetate, sodium phosphate, calcium chloride, magnesium sulfate, and water at the following ions ratio, wt. %:
sodium: 12.4-35.4
potassium: 12.5-15.6
calcium: 0.65-7.785
magnesium: 0.45-4.671
chlorine: 20.4-48.65
phosphate: 3.1-34.4
sulphate: 0.28-13.27
acetate: to 23.27
water: the rest.

2. The saline electrolyte solution according to claim 1, wherein the saline electrolyte solution additionally comprises citric acid or citrate in an amount of from 0.08 to 19.45 wt. %.

3. The saline electrolyte solution according to claim 1, wherein the saline electrolyte solution additionally comprises at least one selected from the group consisting of: vitamins, trace elements, probiotics, prebiotics, metabiotics, amino acids, peptides, lipids, tricarboxylic acids, salts of tricarboxylic acids, and derivatives thereof, and combinations thereof.

4. The saline electrolyte solution according to any one of claim 3, wherein the saline electrolyte solution comprises vitamins, wherein the vitamins are selected from the group consisting of water-soluble vitamins, fat-soluble vitamins, and combinations thereof.

5. The saline electrolyte solution according to claim 1, wherein the saline electrolyte solution additionally comprises medium-chain triglycerides.

6. The saline electrolyte solution according to claim 1, wherein the saline electrolyte solution additionally comprises at least one selected from the group consisting of glucose, fructose, sucrose, and combinations thereof.

7. The saline electrolyte solution according to claim 1, wherein the saline electrolyte solution additionally comprises at least one selected from the group consisting of butanoic acid, monobutyrin, dibutyrin, tributyrin, salts of butanoic acid, salts of monobutyrin, salts of dibutyrin, salts of tributyrin, and combinations thereof.

8. The saline electrolyte solution according to claim 1, wherein the saline electrolyte solution additionally comprises at least one selected from the group consisting of propionic acid, salts of propionic acid, and combinations thereof.

9. The saline electrolyte solution according to claim 1, wherein the saline electrolyte solution additionally comprises at least one selected from the group consisting of acetic acid, salts of acetic acid, and combinations thereof.

10. The saline electrolyte solution according to claim 1, wherein the saline electrolyte solution additionally comprises at least one selected from the group consisting of isobutyric acid, salts of isobutyric acid, and combinations thereof.

11. The saline electrolyte solution according to claim 1, wherein the saline electrolyte solution additionally comprises at least one selected from the group consisting of valerian acid, salts of valerian acid, and combinations thereof.

12. The saline electrolyte solution according to claim 1, wherein the saline electrolyte solution additionally comprises at least one selected from the group consisting of isovaleric acid, salts of isovaleric acid, and combinations thereof.

13. The saline electrolyte solution according to claim 1, wherein the saline electrolyte solution additionally comprises lactic acid, salts of lactic acid, and combinations thereof.

14. The saline electrolyte solution according to claim 1, wherein the saline electrolyte solution additionally comprises at least one selected from the group consisting of maleic acid, salts of maleic acid, and combinations thereof.

15. The saline electrolyte solution according to claim 1, wherein the saline electrolyte solution additionally comprises at least one selected from the group consisting of bacteria, bacterial products, and combinations thereof.

16. The saline electrolyte solution according to claim 15, wherein the saline electrolyte solution comprises at least one selected from the group consisting of bifido-, lacto-, coli-bacteria and bacterial products.

17. The saline electrolyte solution according to claim 2, wherein the saline electrolyte solution additionally comprises at least one selected from the group consisting of: vitamins, trace elements, probiotics, prebiotics, metabiotics, amino acids, peptides, lipids, tricarboxylic acids, salts of tricarboxylic acids, and derivatives thereof, and combinations thereof.

18. The saline electrolyte solution according to any one of claim 17, wherein the saline electrolyte solution comprises vitamins, wherein the vitamins are selected from the group consisting of water-soluble vitamins, fat-soluble vitamins, and combinations thereof.

19. The saline electrolyte solution according to claim 2, wherein the saline electrolyte solution additionally comprises medium-chain triglycerides.

20. The saline electrolyte solution according to claim 1, wherein the saline electrolyte solution additionally comprises gluconate in an amount of from 0.05 to 18.2 wt. %.
